(19) United States
(12) Patent Application Publication
(10) Pub. No.: US 2014/0235618 A1
(43) Pub. Date: Aug. 21, 2014

(54) METHODS FOR TREATMENT OF ATOPIC DERMATITIS AND INFLAMMATORY SKIN DISORDERS

(71) Applicant: Allergan, Inc., Irvine, CA (US)

(72) Inventors: Michael D. Cheung, Mountain View, CA (US); Dinusha Lalwani, Corona, CA (US)

(73) Assignee: Allergan, Inc., Irvine, CA (US)

(21) Appl. No.: 14/183,810

(22) Filed: Feb. 19, 2014

Related U.S. Application Data
(60) Provisional application No. 61/767,544, filed on Feb. 21, 2013.

(51) Int. Cl. A61K 31/55; A61K 9/00

(52) U.S. Cl. CPC A61K 31/55; A61K 9/0014
USPC 514/214.02

(57) ABSTRACT

The present invention provides methods for the treatment of atopic dermatitis and other inflammatory skin disorders. It has been found that topical application of alcaftadine compositions is useful for prophylaxis and symptomatic management of atopic dermatitis and associated symptoms.

### FIELD OF THE INVENTION

The present invention relates generally to compositions and methods useful for treating a variety of dermatological conditions. In particular, the invention relates to alcaftadine compositions and methods for the treatment of atopic dermatitis and other inflammatory skin disorders.

### BACKGROUND OF THE INVENTION

Atopic dermatitis (eczema) is the most common childhood skin disorder in developed countries, characterized by chronically inflamed and intensely pruritic skin. The prevalence rate for atopic dermatitis is 10-12% in children and 0.9% in adults. More recent information examining physician visits for atopic dermatitis in the United States from 1997-2004 estimates that a large increase in office visits for atopic dermatitis occurred. In addition, African-Americans and Asians visit the physician more frequently for atopic dermatitis than whites. Note that this increase involves all diseases under the umbrella of atopic dermatitis and it has not been possible to allocate which classification type has increased so rapidly. Current treatments for atopic dermatitis include histamine H1 receptor antagonists, but these treatments have not proven effective and have sedative properties. It has been found that unlike H1 receptor antagonists, H4 receptor antagonists can demonstrate anti-pruritic and anti-inflammatory properties, but not H1 receptor antagonists.

Atopic dermatitis may be associated with other atopic (immunoglobulin E [IgE]-associated) diseases (e.g., acute allergic reaction to foods, asthma, urticaria, and allergic rhinitis).

The cause of atopic dermatitis is multi-factorial, with genetics, environment, and impaired immune response being the most predominant factors. The role of genetics has been demonstrated in studies of families and twins. Numerous environmental factors have been associated with atopic dermatitis: exposure to allergens, irritants, bacteria, and hard water; socioeconomic status; and large family size There is evidence that atopic dermatitis is a risk factor for childhood asthma, affecting asthma occurrence, severity, and persistence.

Atopic dermatitis is likely related to early IgE production and later allergen/IgE reactivity. The impaired immune response is characterized by activation of T-helper type 2 cells, leading to increased interleukin-4 production, which promotes IgE production.

### SUMMARY OF THE INVENTION

The present invention provides methods for the treatment of atopic dermatitis and other inflammatory skin disorders. It has been found that topical application of alcaftadine compositions is useful for prophylaxis and symptomatic management of atopic dermatitis and associated symptoms.

### DETAILED DESCRIPTION OF THE INVENTION

It is to be understood that both the foregoing general description and the following detailed description are exemplary and explanatory only and are not restrictive of the invention claimed. As used herein, the use of the singular includes the plural unless specifically stated otherwise. As used herein, “or” means “and/or” unless stated otherwise. Furthermore, use of the term “including” as well as other forms, such as “includes,” and “included,” is not limiting. The section headings used herein are for organizational purposes only and are not to be construed as limiting the subject matter described.

It has been found that alcaftadine compositions are useful for prophylaxis and symptomatic management of atopic dermatitis and other inflammatory skin disorders. Alcaftadine is a known H1, H2, and H4 histamine receptor antagonist. Furthermore, it decreases chemotaxis and has been shown to demonstrate eosinophil activation inhibition. Alcaftadine is a white to yellow powder in which no polymorphism has been observed. Alcaftadine has the structure:

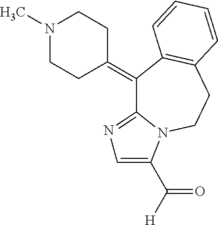 [SMILES: CN1CCC(=C2c3ccccc3CCn3c(C=O)cnc32)CC1]

with a CAS name of 6,11-dihydro-11-(1-methyl-4-piperidinylidene)-5H-imidazo[2,1-b] [3] benzazepine-3-carboxaldehyde.

Pharmaceutically acceptable salts of alcaftadine can be formed from organic and inorganic acids. Suitable acids include, but are not limited to, acetic, 4-acetamido benzoic acid, benzenesulfonic, camphorsulfonic, citric, 2,3:4,6-di-O-isopropylidene-2-keto-L-gulonic acid monohydrate, formic, fumaric, hydrochloric, hydrobromic, lactic, maleic, L-(−)malic, malic, malonic, mandelic, methanesulfonic, naphthalenesulfonic, nitric, oxalic, phthalic, phosphoric, propionic, DL-pyroglutamic, saccharin, salicyclic, succinic, sulfuric, tartaric, trifluoro acetic, L-(+)tartaric, and toluenesulfonic acids.

In certain embodiments of the invention, anti-histamine based compositions are beneficial, including H1/H2/H4 suspensions, such as those marked under the trade name LASTACAFT®.

The term “inflammatory skin disorders” as used herein encompasses human and animal conditions, disorders, or diseases affecting skin. Such skin conditions include, but are not limited to, conditions involving skin inflammation, conditions involving sebaceous glands and hair follicles, conditions characterized by acneiform symptoms, and conditions involving skin dryness, skin thickening, skin scaling, or skin flaking. Skin conditions encompassed by the embodiments of the compositions, products, and methods described herein include, but are not limited to, acne, rosacea, folliculitis, perioral dermatitis, photodamage, skin aging, psoriasis, ichtiosis, treatment of chronic wounds, bed sores, keratosis piralis, scars, including surgical and acne scars, sebaceous cysts, inflammatory dermatoses, post inflammatory hyperpigmentation, xerosis, pruritis, lichen planus, nodular prurigo, eczema, and miliaria.

As used herein, the terms “treatment” or “treating” in reference to a skin condition generally mean “having positive effect on a skin condition” and encompass alleviation of at least one symptom of a skin condition, a reduction in the severity of the skin conditions, or delay, prevention, or inhibition of the progression of the skin condition. Treatment need not mean that the condition is completely cured. A composition or a product useful for treatment of a skin condition, or a method of treating a skin condition, needs only to reduce the severity of a skin condition, reduce the severity of symptoms associated therewith, provide improvement to a patient's quality of life, or delay, prevent, or inhibit the onset of symptoms of a skin condition.

Compositions used in the practice of the present invention are typically in the form of one selected from the group consisting of a gel, emulsion, cream, liquid, paste, lotion, nanoemulsion, microemulsion, reverse emulsion, and liposomal cream.

The methods of the invention employ compositions including semi-solid and gel-like vehicles that include a polymer thickener, water, preservatives, active surfactants or emulsifiers, antioxidants, sunscreens, and a solvent or mixed solvent system. The solvent or mixed solvent system is important to the formation of the microparticulate to dissolved pharmaceutical ratio. The formation of the microparticulate, however, should not interfere with the ability of the polymer thickener or preservative systems to perform their functions.

Polymer thickeners that may be used include hydrophilic and hydroalcoholic gelling agents frequently used in the cosmetic and pharmaceutical industries. Preferably, the hydrophilic or hydroalcoholic gelling agent comprises “CARBOPOL®” (B.F. Goodrich, Cleveland, Ohio), “HYPAN®” (Kingston Technologies, Dayton, N.J.), “NATROSOL®” (Aqualon, Wilmington, Del.), “KLUCEL®” (Aqualon, Wilmington, Del.), or “STABILEZE®” (ISP Technologies, Wayne, N.J.). Preferably, the gelling agent comprises between about 0.2% to about 4% by weight of the composition. More particularly, the preferred compositional weight percent range for “CARBOPOL®” is between about 0.5% to about 2%, while the preferred weight percent range for “NATROSOL®)” and “KLUCEL®” is between about 0.5% to about 4%. The preferred compositional weight percent range for both “HYPAN®” and “STABILEZE®” is between about 0.5% to about 4%.

CARBOPOL® is one of numerous cross-linked acrylic acid polymers that are given the generally adopted name carbomer. These polymers dissolve in water and form a clear or slightly hazy gel upon neutralization with a caustic material such as sodium hydroxide, potassium hydroxide, triethanolamine, or other amine bases. KLUCEL is a cellulose polymer that is dispersed in water and forms a uniform gel upon complete hydration. Other preferred gelling polymers include hydroxyethylcellulose, cellulose gum, MVE/MA decadiene crosspolymer, PVM/MA copolymer, or a combination thereof.

Preservatives may also be used in this invention and preferably comprise about 0.05% to 0.5% by weight of the total composition. The use of preservatives assures that if the product is microbially contaminated, the formulation will prevent or diminish microorganism growth. Some preservatives useful in this invention include methylparaben, propylparaben, butylparaben, chloroxylenol, sodium benzoate, DMDM Hydantoin, 3-Iodo-2-Propylbutyl carbamate, potassium sorbate, chlorhexidine digluconate, or a combination thereof.

Pharmaceuticals for use in all embodiments of the invention include antimicrobial agents, anti-inflammatory agents, antiviral agents, local anesthetic agents, corticosteroids, destructive therapy agents, antifungals, and antiandrogens. In the treatment of acne, active pharmaceuticals that may be used include antimicrobial agents, especially those having anti-inflammatory properties such as dapsone, erythromycin, minocycline, tetracycline, clindamycin, and other antimicrobials. The preferred weight percentages for the antimicrobials are 0.5% to 10%. In the topical treatment of herpes lesions, active pharmaceuticals that may be used include antiviral or local anesthetic agents. A concentration of about 1.0% to 10% by weight is preferred for nucleoside analogues such as acyclovir, famciclovir, penciclovir, valacyclovir, and ganciclovir.

Local anesthetics include tetracaine, tetracaine hydrochloride, lidocaine, lidocaine hydrochloride, dyclonine, dyclonine hydrochloride, dimethisoquin hydrochloride, dibucaine, dibucaine hydrochloride, butambenpicrate, and pramoxine hydrochloride. A preferred concentration for local anesthetics is about 0.025% to 5% by weight of the total composition. Anesthetics such as benzocaine may also be used at a preferred concentration of about 2% to 25% by weight.

Corticosteroids that may be used include betamethasone dipropionate, fluocinolone acetonide, betamethasone valerate, triamcinolone acetonide, clobetasol propionate, desoximetasone, diflorasone diacetate, amcinonide, flurandrenolide, hydrocortisone valerate, hydrocortisone butyrate, and desonide are recommended at concentrations of about 0.01% to 1.0% by weight. Preferred concentrations for corticosteroids such as hydrocortisone or methylprednisolone acetate are from about 0.2% to about 5.0% by weight.

Destructive therapy agents such as salicylic acid or lactic acid may also be used. A concentration of about 2% to about 40% by weight is preferred. Cantharidin is preferably utilized in a concentration of about 5% to about 30% by weight. Typical antifungals that may be used in this invention and their preferred weight concentrations include: oxiconazole nitrate (0.1% to 5.0%), ciclopirox olainine (0.1% to 5.0%), ketoconazole (0.1% to 5.0%), miconazole nitrate (0.1% to 5.0%), and butoconazole nitrate (0.1% to 5.0%). For the topical treatment of seborrheic dermatitis, hirsutism, acne, and alopecia, the active pharmaceutical may include an antiandrogen such as flutamide or finasteride in preferred weight percentages of about 0.5% to 10%.

In some embodiments of the invention, the methods employ compositions including solubilizing agents selected from alcohols, glycols, esters, ethers, or silicones. Such solubilizing agents include, but are not limited to, PEG 400, lactic acid, dimethyl isosorbide, propylene glycol, propylene carbonate, hexylene glycol, isostearyl alcohol, isopropyl myristate, ethanol, or triglycerides.

In certain embodiments, the solubilizing agent is propylene glycol.

In certain embodiments, the second solubilizing agent is propylene carbonate.

In certain embodiments, the second solubilizing agent is ethanol.

In some embodiments of the invention, the compositions further include methyl paraben.

In other embodiments, the compositions further include carbopol 980.

In some embodiments of the invention, the compositions further include a neutralizing agent. In certain embodiments, the neutralizing agent is NaOH or triethanolamine. Use of a neutralizing agent results in compositions typically having a pH from 5.5 to 6.5.

In some embodiments of the invention, the compositions further include a chelating agent. In some embodiments, the chelating agent is ethylene diamine tetraacetic acid (EDTA). EDTA is typically present in the compositions from 0.02% w/w to 0.04% w/w. In certain embodiments, EDTA is present in the compositions at 0.03% w/w.

What is claimed is:

1. A method for treating an inflammatory skin disorder comprising administering to a subject in need thereof a therapeutically effective amount of a composition comprising a compound having the structure: 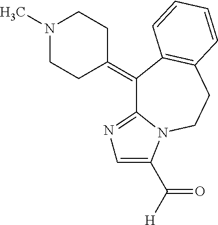 [SMILES: CN1CCC(=C2c3ccccc3CCn3c(C=O)cnc32)CC1]
or a pharmaceutically acceptable salt thereof.

2. The method of claim 1 wherein the skin disorder is selected from the group consisting of atopic dermatitis, eczema, acne vulgaris, rosacea, treatment of chronic wounds, bed sores, keratosis piralis, sebaceous cysts, inflammatory dermatoses, post inflammatory hyperpigmentation, eczema, xerosis, pruritis, lichen planus, nodular prurigo, dermatitis, and miliaria.

3. The method of claim 1 wherein the condition is atopic dermatitis.

4. The method of claim 1, wherein the composition further comprises a pharmaceutically acceptable carrier.

5. The method according to claim 4, wherein the carrier comprises at least one agent selected from the group consisting of a moisturizing agent, a pH adjusting agent, a deodorant agent, a fragrance, a hair-conditioning agent, a chelating agent, a preservative, an emulsifier, a thickener, a solubilizing agent, a penetration enhancer, an anti-irritant, a colorant, a surfactant, and combinations thereof.

6. The method according to claim 5, wherein the moisturizing agent is selected from the group consisting of guanidine, glycolic acid, glycolate salts, aloe vera, allantoin, urazole, polyhydroxy alcohols, propylene glycol, butylene glycol, hexylene glycol, polyethylene glycols, sugars, starchs, alkoxylated glucose, hyaluronic acid, lactamide monoethanolamine, acetamide monoethanolamine, and combinations thereof.

7. The method according to claim 1, wherein the composition has a pH from about 5 to about 6.5.
